(19) United States
(12) Patent Application Publication
(10) Pub. No.: US 2025/0336062 A1
(43) Pub. Date: Oct. 30, 2025

(54) HEATMAP BASED FEATURE PRESELECTION FOR RETINAL IMAGE ANALYSIS

(71) Applicant: OPTINA DIAGNOSTICS, INC., Montreal (CA)

(72) Inventors: Claudia CHEVREFILS, Montreal (CA); Jean-Sebastien GRONDIN, Montreal (CA); David LAPOINTE, Montreal (CA); Sam OSSEIRAN, Montreal (CA); Jean-Philippe SYLVESTRE, Montreal (CA); Adrian TOUSIGNANT DURAN, Montreal (CA)

(21) Appl. No.: 19/038,485

(22) Filed: Jan. 27, 2025

Related U.S. Application Data
(63) Continuation of application No. PCT/IB2023/057724, filed on Jul. 28, 2023.
(60) Provisional application No. 63/392,957, filed on Jul. 28, 2022.

(51) Int. Cl. G06T 7/00

(52) U.S. Cl. CPC G06T 7/0012; G06T 7/97; G06T 2207/10048; G06T 2207/20172; G06T 2207/30041

(57) ABSTRACT

Systems and methods described herein process retinal image data and select features that are most useful in the detection of disease. The systems/methods generate heatmaps indicating the discriminative power of various spatial/spectral information and use the heatmaps for feature selection and training of ML models.

### PRIORITY

This application is a continuation of PCT Patent Application No. PCT/IB2023/057724, entitled “HEATMAP BASED FEATURE PRESELECTION FOR RETINAL IMAGE ANALYSIS”, filed on Jul. 28, 2023, which claims priority to U.S. provisional patent application 63/392,957, filed Jul. 28, 2022, the entirety of which is herein incorporated by reference.

### BACKGROUND

The retina is a thin layer of tissue located at the back of the eye that is part of the fundus. The retina is highly vascularized, meaning it contains a dense network of blood vessels, and it is part of the central nervous system. The retina is also largely transparent, allowing light to pass through and reach the photoreceptors. This transparency makes it possible to non-invasively capture detailed images that include the blood vessels and features of the central nervous system. These images can provide valuable information about the health of the vascular and nervous systems.

Multispectral and hyperspectral fundus imaging techniques have increasingly been used for diagnostic and other purposes. These techniques involve capturing images of the fundus and retina at different wavelengths of light, where the different wavelengths provide different spectral responses based on the features of the blood vessels and other structures in the fundus. These wavelength-specific images allow for more detailed analyses of the fundus/retina, including the detection and diagnosis of a wide range of ocular and systemic diseases, such as diabetes, cardiovascular diseases, neurological disorders like Alzheimer's disease (AD), organ-specific diseases, and the like.

Multispectral or hyperspectral cameras capture a large amount of spatial and spectral data by taking a series of images at different wavelengths (e.g., using bandpass filters or other techniques). In fundus imaging applications, the captured reflectance spectrum is influenced by the molecular content (e.g., hemoglobin, melanin), cellular arrangement (e.g., capillaries, nerve fiber layer), and density/thickness (e.g., neurodegeneration) of the tissue at the various wavelengths of the spectrum. The large amount of captured data can make analysis for diagnostic purposes difficult. Accordingly, techniques for processing the image data to improve diagnostics are highly desirable.

### SUMMARY

According to some embodiments of the present disclosure, a computer-implemented method, system configured to perform the method, and computer-readable medium including instructions for carrying out the method is disclosed. The method may include receiving a plurality of retinal images corresponding to a plurality of patients. The method may further include modifying a subset of the plurality of retinal images so that each of the plurality of retinal images shares an orientation. The method may further include generating, based on the padded plurality of retinal images, a stack of retinal images for further analysis, where each retinal image of the stack of retinal images comprises a defined number of pixels, wherein each retinal image of the stack of retinal images is associated with at least one reference label indicating the presence or absence of a medical condition. The method may further include, for each pixel of the defined number of pixels: calculating a first distribution value for the corresponding pixel, wherein the first distribution value is based on pixel values from a first subset of retinal images associated with a positive reference label for the medical condition; calculating a second distribution value, wherein the second distribution value is based on pixel values from a second subset of retinal images associated with a negative reference label for the medical condition; and calculating a comparison metric for the corresponding pixel based on the first distribution value and the second distribution value. The method may further include generating a heat map based on the comparison metric for each pixel of the defined number of pixels. The method may further include calculating a discriminative power of the heat map for detecting the medical condition.

In embodiments, the heat map is a first heat map associated with a first spectral range and a first texture type, the method further comprising: generating a plurality of heat maps, wherein each heat map is associated with a spectral range and a texture type, wherein the plurality of heat maps includes the first heat map; calculating a discriminative power for each of the plurality of heat maps; and ranking the plurality of heat maps based on the corresponding discriminative power of each of the plurality of heat maps. In some of these embodiments, the method further comprises selecting one or more features to train a machine learning model for detecting the medical condition based on the ranking of the plurality of heatmaps.

In embodiments, calculating the discriminative power of the heat map comprises calculating the mean of each comparison metric of the heat map. Additionally or alternatively, calculating the discriminative power of the heat map comprises calculating the mean of each squared comparison metric of the heat map. Additionally or alternatively, calculating the discriminative power of the heat map comprises calculating the mean of a sub-set of top-k comparison metrics of the heat map.

In embodiments, modifying the subset of the plurality of retinal images comprises flipping the subset of retinal images that correspond to a left or right eye so that they share the orientation of a right or left eye. Additionally or alternatively, modifying the subset of the plurality of retinal images comprises padding each retinal image to center an optical nerve head within each of the plurality of retinal images.

In embodiments, the calculating steps are performed as part of a Student's t-test. Additionally or alternatively, generating the stack of retinal images further comprises performing non-linear image registration to align retinal anatomical landmarks associated with each of the plurality of patients.

The method may include generating a stack of retinal images corresponding to a plurality of patients, wherein each retinal image of the stack of retinal images is aligned and comprises a defined number of pixels, wherein each retinal image of the stack of retinal images is associated with at least one reference label indicating the presence or absence of a medical condition. The method may further include generating a first heat map for a first subset of images from the stack of retinal images by: for each pixel of the defined number of pixels of the first subset of images: calculating a first distribution value for the corresponding pixel, wherein the first distribution value is based on pixel values from a first subset of retinal images associated with a positive reference label for the medical condition; calculating a second distribution value for the corresponding pixel, wherein the second mean pixel value is based on pixel values from a second subset of retinal images associated with a negative reference label for the medical condition; and calculating a comparison metric for the corresponding pixel based on the first distribution value and the second distribution value; and generating the heat map for the first subset of images based on the comparison metric for each pixel of the defined number of pixels. The method may further include generating additional heat maps for additional subsets of images from the stack of retinal images to yield a plurality of heat maps, wherein the plurality of heat maps comprises the first heat map. The method may further include determining a discriminative power of each heat map of the plurality of heat maps. The method may further include ranking the plurality of heat maps based on the corresponding discriminative power of each of the plurality of heat maps.

In embodiments, the method may further include selecting a plurality of features corresponding to pixels of the heat maps that have high discriminative power for the medical condition; and training an ML model to predict the absence or presence of the medical condition based on the selected plurality of features.

In embodiments, calculating the discriminative power of each heat map comprises, for each heat map, calculating the mean of each comparison metric. Additionally or alternatively, calculating the discriminative power of each heat map comprises, for each heat map, calculating the mean of each squared comparison metric of the heat map. Additionally or alternatively, calculating the discriminative power of the heat map comprises calculating the mean of a sub-set of top-k comparison metrics of the heat map.

In embodiments, generating the stack of retinal images comprises flipping a subset of retinal images that correspond to a left or right eye so that they share the orientation of a right or left eye. Additionally or alternatively, generating the stack of retinal images comprises padding each image of the stack of retinal images to align an optical nerve head within each image.

In embodiments, the calculating steps are performed as part of a Student's t-test. Additionally or alternatively, generating the stack of retinal images further comprises performing non-linear image registration to align retinal anatomical landmarks associated with each of the plurality of patients.

The method may comprise generating a stack of retinal images corresponding to a plurality of patients, wherein each retinal image of the stack of retinal images is aligned, wherein each retinal image of the stack of retinal images is associated with at least one reference label indicating the presence or absence of a medical condition. The method may further comprise generating texture measures for the stack of retinal images. The method may further comprise applying a plurality of anatomical masks to the stack of retinal images. The method may further comprise selecting spectral regions. The method may further comprise generating a plurality of features, wherein each feature corresponds to a particular texture measure, anatomical mask, and spectral region. The method may further comprise generating values for each of the plurality of features from the stack of retinal images to yield a feature grid comprising feature values for each of the plurality of features. The method may further comprise generating a heatmap based on the feature grid and a classification label indicating the presence or absence of a medical condition. The method may further comprise measuring a discriminative power of the heatmap.

In embodiments, the method may further comprise selecting a plurality of features from the heat map that have high discriminative power for the medical condition; and training an ML model to predict the absence or presence of the medical condition based on the selected plurality of features. Additionally or alternatively, the anatomical mask corresponds to one of blood vessels, an optic nerve head, or a background retina, among other features described herein. Additionally or alternatively, the texture measures comprise one or more of a contrast measure, a homogeneity measure, an energy measure, or a correlation measure, among other texture measures described herein.

The method may comprise receiving a stack of retinal images corresponding to a plurality of patients, wherein each retinal image of the stack of retinal images is associated with at least one reference label indicating the presence or absence of a medical condition. The method may further comprise selecting a plurality of features based on the stack of retinal images, wherein each feature is associated with at least one of an anatomical mask applied to the retinal images, a spectral region, or a texture measure. The method may further comprise generating a feature heatmap that indicates a discriminative power of each of the plurality of features with respect to the reference label. The method may further comprise generating a plurality of image heatmaps, wherein each of the plurality of image heatmaps is generated using a pixel-wise statistical test to determine the discriminative power of each pixel of a subset of the stack of retinal images. The method may further comprise selecting a plurality of top-k features based on a corresponding discriminative power of each feature, wherein each feature corresponds to a feature of the feature heatmaps or a pixel of the image heatmaps. The method may further comprise training a machine learning model to predict the presence or absence of the condition based on the plurality of top-k features.

These features, along with many others, are discussed in greater detail below.

### BRIEF DESCRIPTION OF THE DRAWINGS

The present disclosure is described by way of example and not limited in the accompanying figures in which like reference numerals indicate similar elements and in which:

FIG. 1 illustrates an example environment including a platform and other devices for carrying out the techniques described herein.

FIG. 2 illustrates an example storage comprising various data generated and used by the techniques described herein.

FIG. 3 illustrates an example method for heatmap based feature preselection according to the techniques described herein.

FIGS. 4A-C illustrates example visualizations of heatmaps generated according to the techniques described herein.

FIG. 5 illustrates an example data flow for heatmap based feature preselection according to techniques described herein.

FIG. 6 illustrates another example method for heatmap based feature preselection according to the techniques described herein.

FIG. 7 illustrates another example visualization of a heatmap according to the techniques described herein.

FIG. 8 illustrates an example platform for carrying out the techniques described herein.

FIG. 9 is a representation of a hyperspectral retinal camera along with a schematic representation of a hyperspectral retinal dataset, in accordance with non-limiting embodiments of the present technology;

FIG. 10 is a schematic representation of a reflectance spectrum from each pixel of a hyperspectral retinal image, in accordance with non-limiting embodiments of the present technology;

FIG. 11 is a schematic representation of a computer-implemented method allowing extraction of features from retinal images, in accordance with non-limiting embodiments of the present technology;

FIG. 12 is a schematic representation of a workflow allowing creation of tests, in accordance with non-limiting embodiments of the present technology;

FIG. 13 is a schematic representation of a computer-implemented system operating a method of processing retinal images, in accordance with non-limiting embodiments of the present technology; and

FIG. 14 illustrates a flow diagram showing operations of a method for processing retinal images, in accordance with non-limiting embodiments of the present technology.

### DETAILED DESCRIPTION

Techniques described herein involve processing retinal image data and selecting features that are most useful in the detection of disease. Hyperspectral imaging techniques generate vast amounts of data, which creates challenges for finding and selecting the most relevant data for analysis and detection of disease, especially for automated methods like machine learning. The techniques described herein generate heatmaps for feature selection and training of ML models. The heatmaps are generated to emphasize the most significant and predictive data for detection of a medical condition, which can be automatically used to select the most relevant features for both training and inference using machine learning models.

Techniques described herein beneficially use both spatial and spectral information to provide a variety of heatmaps that clearly indicate which spatial and spectral features are most discriminative for a particular condition. For example, the heatmap-based feature pre-selection described herein may leverage pixel-wise statistical tests to generate heatmaps for various spectral information divided into at least two groups of interest (e.g., a group with the condition and a group without the condition), thereby automatically identifying spatial anatomical regions most relevant to the classification task, where the most relevant spatial anatomical regions may vary by spectral range. The techniques described herein may then use the heatmaps to infer a ranking of feature importance, thereby providing an automated approach to reducing input dimensions so as to identify the best candidate features for downstream ML algorithms.

Accordingly, the heatmap-based feature pre-selection described herein provides a novel, systematic, and statistically guided method/system for processing retinal imaging data to better train and run ML models. By incorporating both spectral and spatial information, the systems/methods described herein provide a more comprehensive approach to feature selection, advancing the state-of-the-art in retinal disease diagnosis and prognosis. These and other benefits will be apparent from the detailed disclosure below.

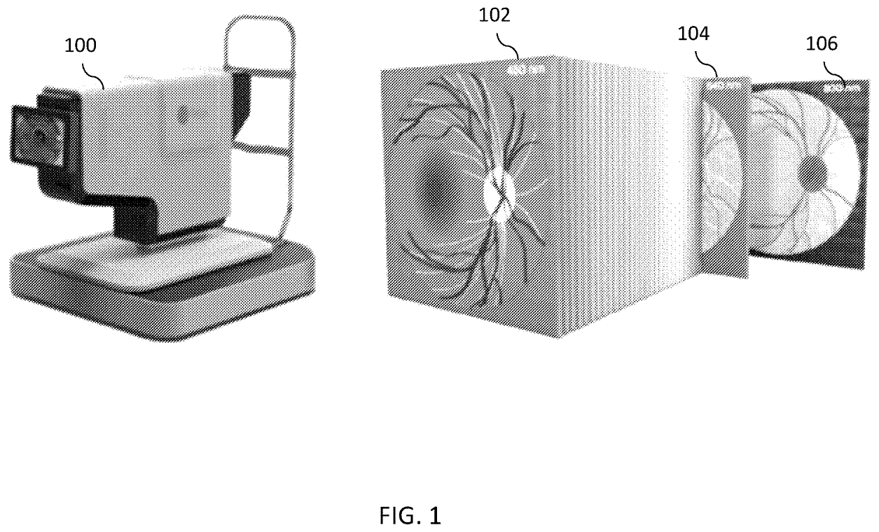

FIG. 1 illustrates an example environment 100 including a plurality of devices that may be used for carrying out the techniques described herein. The environment 100 may include a feature preselection and training platform 110, a plurality of hyper-spectral retinal cameras 120A-N, one or more user devices 130A-N, and one or more analysis systems 140A-N in communication via one or more networks 150. It will be appreciated that the network connections shown are illustrative and any means of establishing communications links between the various devices and systems may be used, including direct cabling or other peer-to-peer communications instead of or in addition to the one or more networks 150. The existence of any of various network protocols such as TCP/IP, Ethernet, FTP, HTTP and the like, and of various wired or wireless communication technologies such as USB, GSM, CDMA, Wi-Fi, and LTE, is presumed, and the various computing devices described herein may be configured to communicate using any of these communication protocols or technologies.

The feature preselection and training platform 110 may store various data (including computer-executable instructions) for performing the various functions described herein. In general, the feature preselection and training platform 110 may provide functionality for processing image data received from the various retinal cameras 120A-N as described herein and training one or more machine learning (ML) models. For example, the feature preselection and training system may be configured to receive hyperspectral cubes comprising retinal scans and/or various patient metadata (e.g., morphological features, age, gender, etc.), generate texture images and/or other types of images based on the hyperspectral images as described herein, perform various statistical analyses (e.g., to generate heat maps as described herein), perform ML training, and perform any of the other tasks herein based on image data received from the retinal cameras 120. The platform 110 may store various data to facilitate the operations described here, including statistical analysis libraries 112, ML model training libraries 114, and/or other such data that may be leveraged by the feature preselection and training platform 110. The feature preselection and training platform 110 may also store various images captured by the retinal cameras and data derived therefrom, including hyperspectral data cubes, texture images, and heat maps, in the storage 116. In embodiments, the image data may be associated with metadata indicating various information such as an identifier of the corresponding retinal camera that captured the data, corresponding patient data and/or other metadata, etc. In embodiments, different retinal cameras 120 may be different types of camera (e.g., different manufacturer/make/model/etc.) and/or may capture different types of data (e.g., multispectral cubes vs. hyperspectral cubes).

In embodiments, although the storage 116 is illustrated as being a component of the feature preselection and training platform 110, in other embodiments the storage 116 may be a part of other systems. For example, any data described herein may be stored in cloud storage, stored at a separate server device connected to the feature preselection and training platform 110, and/or the like.

The user devices 130A-N may be used by various users to interact with the feature preselection and training platform 110, view image and heatmap data, view ranked heatmaps, control ML training based on the heatmaps, edit various data, generate analyses, and/or the like. In embodiments, the feature preselection and training platform 110 may restrict access to the image data (e.g., in accordance with medical data privacy laws or other rules that may vary by jurisdiction) to certain user devices based on authorization credentials provided by a user using the user device 130. The user devices 130A-N may include mobile devices (e.g., smartphones, laptops, tablets), wearable devices (e.g., smartwatches), other computing devices (e.g., desktop computers, servers), and/or any other devices that may access and interact with the feature preselection and training platform 110.

The analysis systems 140A-N may include automated systems that may use the image data, heat maps, ML models, and/or other data described herein to perform automated analyses. For example, the analysis systems may receive and/or execute trained machine learning models or use other automated approaches to perform automated analysis, generate diagnostic information, and/or the like. In embodiments, the automated analyses and/or diagnostics generated by the analysis systems may be provided by the analysis systems 140A-N to the user devices 130A-N. In embodiments, the analysis devices 140A-N may require authorization from a particular user device 130 to make the analyses and/or diagnostics available to the user device 130.

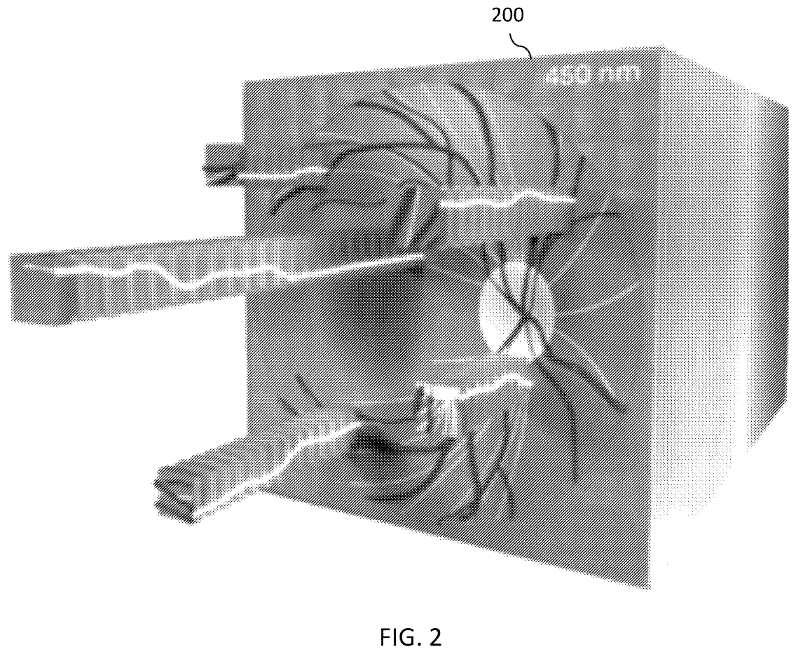

FIG. 2 illustrates example data that may be stored in the storage 116 for use by the platform 110 as described in more detail below. The data may include a hyperspectral image data set 200 comprising a plurality of hyperspectral patient images and metadata, a texture data image set 210 comprising texture images and metadata, where the texture images and data may be generated based on the hyperspectral patient images and metadata, and a plurality of heatmaps 220 that may be generated as described in more detail below. As illustrated, the hyperspectral image data set 200 may include sets of patient 1-N images 202A-N, where each set of patient images 202A may include a plurality of hyperspectral images corresponding to various wavelengths or bands 2. Each set of patient images 202 may be associated with corresponding metadata 204, which may include patient information, diagnostic information (e.g., whether the patient was diagnosed with a particular condition or not), and other information.

In some cases, each set of patient images 202 and/or metadata 204 may be structured as a hyperspectral cube comprising a series of retinal images and corresponding metadata for each image. Alternatively, set of patient images 202 and/or metadata 204 may be multispectral cube (e.g., depending on the type and settings of the retinal camera used to capture the images). Each set of patient images 202 (whether structured as a cube or not) may include a large number of images (e.g., tens, hundreds, or thousands) that may be captured across a range of wavelengths, which may overlap in the case of hyperspectral images. Alternatively, if the images are multispectral images, the set of images for each patient may include a smaller number of narrowband images (e.g., a few dozen or fewer) that may be captured across a different range of wavelengths, which may often include spectral gaps such that the cube includes spaced spectral bands.

The series of images 202 for each patient may include a first image corresponding to a first wavelength λ1, a second image corresponding to a second wavelength λ2, and so on. Although an image may be referred to as corresponding to a particular wavelength, in practice each image in the cube may include information captured within a specific band of wavelengths that includes the particular wavelength (e.g., the term “wavelength” may refer to a representative wavelength within a band). The bands may be relatively broad and/or non-overlapping (e.g., for multispectral cubes) or relatively narrow and/or contiguous or overlapping (e.g., for hyperspectral cubes).

In some cases, the hyperspectral image data set 200 may be normalized, registered, and/or otherwise preprocessed before it is used in the various methods described below. For example, each patient's images may have been aligned to remove any discrepancies in the orientation of the patient with respect to the retinal camera (e.g., if the patient moved while the images were being capture) and/or normalized to remove image artifacts, camera artifacts, light leak from camera or other lighting sources, distortions caused by the retina or camera, various spectral influences, and/or other influences that may reduce the effectiveness of analyses based on the image data. These and/or other preprocessing operations may be carried out by the platform 110 and/or some other system (e.g., retinal cameras 120, user devices 130, analysis systems 140, etc.).

The storage 116 may further include a texture image data set 210 including several sets 212, where each set includes a plurality of texture images 214 and associated metadata 216. The texture images 214 may be generated by the platform 110 for use in creating heatmaps as described in more detail below. Additionally or alternatively, the texture images 214 may be generated by another device and received by the platform 110 for storage at storage 116. The generation of various types of texture images is described in U.S. Pat. No. 10,964,036 (herein “the '036 Patent”), which is hereby incorporated by reference in its entirety. As shown in the figure, the texture image data set 210 may store various types of texture images (e.g., 4 different types in the illustrated example) in various sets 212. Each set 212 of texture images 214 may include images generated based on the patient 1-N images 202A-N, where each texture image 214 may correspond to a particular patient and wavelength. In other words, if the hyperspectral image data set 200 includes images for a number m of patients with a number n of different wavelength images per patient, then each set of texture images 212 may include m*n images. Each different type of texture image may be generated using gray level co-occurrence matrix (GLCM), as described in the '036 Patent. For example, as described in the '036 Patent, a first type of texture image may be a contrast image, a second type of texture image may be a homogeneity image, a third type of image may be a correlation image, and a fourth type of image may be an energy image. However, other techniques may be used to generate the texture images.

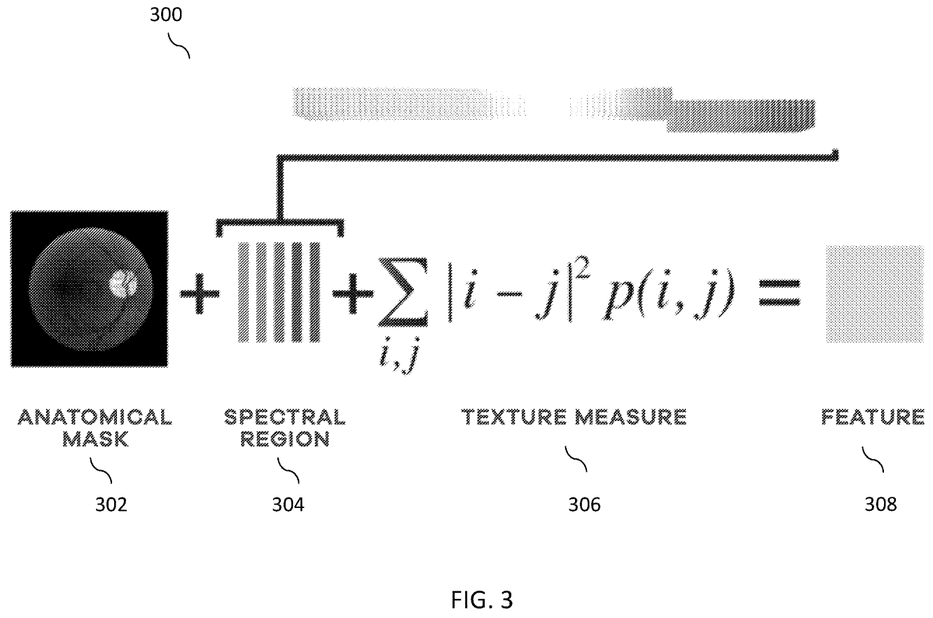

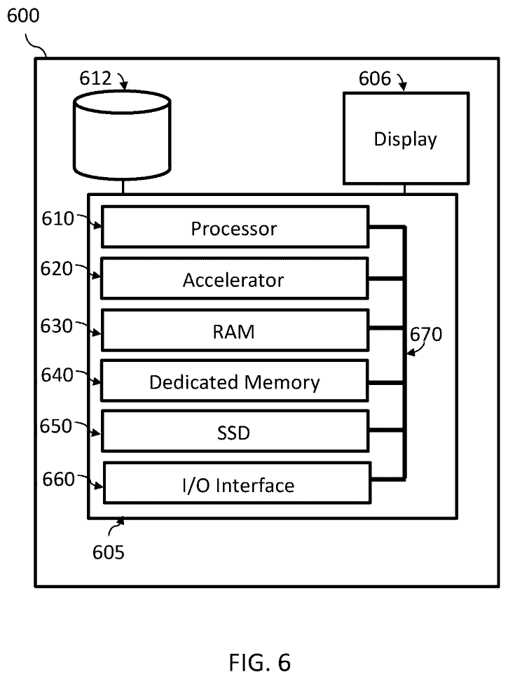

The storage 116 may further include various types of heatmaps 220 generated according to the various methods and techniques described herein. For example, the different types of texture heatmaps 222A-D may be generated based on the texture image data set as described in the method of FIG. 3. Additionally or alternatively, the storage 116 may include feature heatmaps 224 that may be generated according to the method of FIG. 6. The storage 116 may also include other heatmaps 226, such as heatmaps generated directly from the hyperspectral image data set, as described in more detail below. Any or all of the heatmaps may be used for heatmap based feature preselection as described herein.

FIG. 3 illustrates an example method for generating heatmaps and using the heatmap for feature preselection and training of a machine learning model. The method of FIG. 3 may be carried out by the platform as an example method of generating various heatmaps and using the heatmaps for feature preselection and ML training. At step 302, the platform 110 may retrieve a plurality of hyperspectral images from a hyperspectral image dataset 200. As described above, this dataset may include images from numerous patients, (including left and right eyes), where each patient may be associated with a number of hyperspectral images for different wavelengths. In some cases, the images may have been captured from different retinal cameras. The images may have been normalized and/or registered and/or may be normalized and/or registered by platform 110 if they have not yet been normalized and/or registered.

Each image within the hyperspectral image dataset may be associated with a diagnosis label, which may be used as a classification label. In several of the examples described herein, the diagnosis/classification labels may be binary (e.g., indicating a particular condition is present or absent). However, it should be understood that there may be a greater number of classifications (e.g., different stages or severities of a condition) and/or the data set may include a continuous label (e.g., a percent likelihood that the patient has a given disease and/or a score for the severity of a condition). Additionally or alternatively, each image may be associated with multiple classification labels, such as when an ML model is being trained to predict multiple diseases or condition. In any of these embodiments, the diagnoses may have been generated manually and/or via any automated mechanism. A binary classification label for each image may indicate a positive or negative diagnosis for the corresponding patient.

At step 304, the platform 110 may process the hyperspectral image dataset 200 of the previous step to generate one or more texture images for a texture image dataset 210 (e.g., if the heatmaps are being generated based on texture images). Additionally or alternatively, the platform 110 may perform various other types of preprocessing to generate images based on the hyperspectral image dataset 200 (e.g., filtering to enhance contrast). For example, various other types of preprocessed images (e.g., filtered images) may be used to generate heat maps. It should also be noted that in some cases, as discussed in more detail below, the platform 110 may generate heatmaps based on the images of the data set 200 without generating any texture images and/or otherwise performing any preprocessing (e.g., the platform may use raw hyperspectral images to generate heat maps). Thus, in some cases step 304 may be optional.

In embodiments that use texture images to generate heatmaps, the platform 110 may generate a texture image dataset 210 comprising multiple texture sets 212 of images, wherein the images 214 within each set 212 correspond to a different texture type. For instance, in the illustrated embodiment, there are four distinct texture types resulting in four corresponding sets of texture images within the dataset. Each of these sets may contain a multitude of images, each image generated using a different texture image generation technique based on corresponding hyperspectral images that are distributed across different wavelength ranges. Each texture image may be associated with a single patient.

Each texture image may be associated with corresponding metadata, which includes, but is not limited to, diagnosis and/or other classification data. Thus, each texture image may be associated with a positive or negative label based on the corresponding patient image(s). The textures in the example embodiment include a contrast texture, a homogeneity texture, an energy texture, and a correlation texture. However, these texture types are merely illustrative, and the platform may accommodate other texture types.

At step 306, the platform 110 may perform processing of at least some of the texture image dataset (e.g., a first subset corresponding to a given heat map type, such as a texture and wavelength combination) and/or any other images that are used to generate heatmaps (e.g., preprocessed hyperspectral images or raw hyperspectral images) to orient the images. For example, the platform may reorient half of the subset of images using a horizontal flip technique to cause the images taken of the patients' right eyes to match the orientation of the images of the left eyes. As will be appreciated, the platform 110 may either flip images from the left eye to align with those from the right eye, or vice versa.

Alternatively, in other embodiments, the platform 110 may employ different reorientation strategies depending on the initial orientation of the images. For example, if the initial orientation of the images differs, the platform 110 may use a vertical flip or another type of warping for alignment. In any case, at step 306 the platform 110 processes a subset of the images to achieve a uniform orientation across the images, which may entail aligning all images along the horizontal axis or any other axis that distinguishes images taken from the left eye from those taken from the right eye. Consequently, the platform 110 synchronizes the orientations of all images, thus improving uniformity and consistency across the dataset.

At step 308, the platform 110 may pad some or all of the images that were oriented in the previous step. The padding may be added such that, for each image, the optical nerve head (ONH) is precisely centered within the padded image frame. Thus, the platform 110 may align the ONHs for each patient, thereby causing a corresponding alignment of the pixel data of the different retinal images.

Although step 308 may include the platform 110 padding each image, it may additionally or alternatively include the platform 110 performing other types of image warping to achieve a desired alignment. For instance, the platform may use various types of image manipulation techniques to center the ONH within the image frame. Additionally or alternatively, the platform 110 may pad or otherwise manipulate each image such that the ONH is aligned to some other reference point instead of the center of the image.

The platform 110 may pad the images to align the ONH by inserting blank, default, or other “dummy” pixels that pad out the image to create the desired centering or other alignment. Via this process, the platform 110 may cause the image data for the various images to be positioned uniformly with respect to the ONH, thus making it easier to compare different images and providing a consistent and standard alignment across the entire dataset.

In embodiments, as shown in FIG. 3, the platform 110 may perform steps 306 and 308 may for each subset of the image dataset. For example, if the platform 110 is generating 24 heat maps, each one corresponding to a given texture type and wavelength range (e.g., with 4 texture types and 6 wavelength ranges per texture type), then the platform 110 may repeat steps 306-308 24 times (once per different subset) to account for each different texture type and wavelength range combination. The platform 110 may perform the same orientation and/or alignment steps for all texture types and all spectral ranges, thus effectively creating a single comprehensive stack of all patient images. The stack of images may include multiple images for each of a plurality of patients (including both left and right eye images), where the multiple images for each patient correspond to multiple texture types and/or multiple wavelengths. In other words, the number of images in the stack may be x*y*z, where x is the number of patients, y is the number of texture types, and z is the number of wavelength ranges.

Each image within the image stack is associated with a classification label. The platform 110 may extract the label from the image metadata 216. The label may denote whether the image corresponds to a positive or negative reference classification (or a more complex classification in some examples).

At step 310, the platform 110 may optionally perform one or more image registration techniques on the resultant image stack to further improve the alignment of the image pixel data across the various images. In particular, the platform 110 may perform the image registration to align various retinal anatomical landmarks across all patients. The retinal anatomical landmarks may include, but are not limited to, retinal arteries, veins, macula, fovea, and the like.

The platform 110 may use various tools and/or algorithms to perform the image registration, including non-linear image registration, intensity-based image registration, and/or or feature-based image registration. As one non-limiting example, the platform may employ B-spline-based nonrigid image registration. In this technique, the platform 110 may use a B-spline transformation model to build a mathematical representation of the deformation field, mapping the anatomical landmarks of one image onto the corresponding landmarks in another image.

Alternatively, the platform 110 may use the Demons algorithm, which is a fast registration method that may be used for registering images. The platform 110 may use the Demons algorithm to estimate displacement fields by iteratively minimizing the difference between the deformed source image and the target image.

Alternatively, the platform 110 may use a Thin Plate Spline (TPS) algorithm for image registration. The platform 110 may use the TPS algorithm to construct a smooth mapping from one image to another by minimizing the bending energy of the transformation, thus causing the landmarks in the source image to precisely match their corresponding landmarks in the target image.

Regardless of the specific registration technique used, the platform 110 may use the image registration process to align the retinal anatomical landmarks across all patients. This alignment may improve the platform's ability to perform precise comparison of different images and may improve the accuracy of subsequent analyses or classifications performed on the dataset. This step, although optional, may provide an additional layer of standardization and accuracy for further improving the dataset prior to generating statistical heat maps.

At step 312, the platform 110 may perform a pixel-wise statistical comparison of the various reference label groups of images corresponding to a particular heat map type (e.g., texture type and wavelength range). Here, the platform 110 may generate a reference class label vector based on the class labels for each image and use the reference class label vector to create at least two comparison groups for each subset of images. For instance, the comparison groups may be a positive diagnosis group and a negative diagnosis group. In some cases, the platform may create more than two groups, for example based on a reference label indicating one of multiple diagnoses (e.g., different severities or stages of a condition). However, the example description provided herein will continue by assuming a binary classification (e.g., a positive or negative diagnosis, creating two comparison groups) with the understanding that other classification schemas are within the scope of the present disclosure.

To conduct the pixel-wise statistical comparison across the two groups of images for each subset of images (e.g., one subset per heat map type), the platform 110 may employ various statistical methods. For example, the platform may use a Student's t-test to calculate a distribution value for each group (positive and negative) and then examine statistical differences for each corresponding pixel between the two distribution groups based on the distribution values. The platform 110 may perform the pixel-wise statistical comparison by comparing the distributions of the two groups of pixel values for a set of first pixels (where each first pixel is a corresponding pixel taken from a different image) to generate a comparison metric for a first pixel, comparing the distributions of the two groups of pixel values for a set of second pixels to generate a comparison metric for the second pixel, and so on. In other words, the pixel-wise comparison may be a comparison of corresponding pixel values across the stack of images. Using the test, the platform 110 generates a single image frame, referred to herein as a heatmap 222. In the generated heatmap 222, each pixel represents a p-value or another statistical comparison metric that measure the significance of the pixel for determining the classification label. For example, a first pixel of the heatmap includes the comparison metric generated based on the set of first pixels across the image stack, a second pixel of the heatmap includes the comparison metric generated based on the set of second pixels across the image stack, and so on. Again, the platform 110 may generate the comparison metric using a pixel-wise t-test or any other statistical comparison that evaluates the significance of each pixel for the reference class label. Accordingly, the platform 110 may identify which pixels of the heatmap hold the most statistical relevance for a given diagnosis.

As alternatives to the Student's t-test, other statistical methods used by the platform 110 may include a Jensen-Shannon divergence, a Chi-square test, a Mann-Whitney U test, or a Wilcoxon signed-rank test, among other such tests or methods. The platform 110 may use any of these or other statistical methods to perform a pixel-by-pixel statistical comparison, thereby determining the discriminatory power of each pixel for the different distributions and encoding it via a heatmap.

As shown in FIG. 3, the platform 110 may perform step 312 multiple times, with each iteration generating a different heat map 222. Thus, for example, the platform 110 may repeat step 312 for different combinations of texture image type and spectral range to generate multiple significance heatmaps (e.g., y*z heatmaps, where y is the number of texture types and z is the number of wavelength ranges), with each heatmap corresponding to a particular texture image type and spectral range.

Although the platform 110 may generate texture heatmaps 222 as described above, the platform 110 may also or alternatively generate other heatmaps 226, such as heatmaps based on non-texture images, including hyperspectral images from the hyperspectral data set 200. For example, the platform 110 may generate a stack of patient images 202 and perform the pixel-wise statistical test on various wavelength-based subsets of the patient images, where each wavelength-based subset may include images for multiple patients that are divided into groups using the classification label. In this example, the platform 110 may generate z other heatmaps 226 (where z is the number of wavelength-based subsets of patient images). Thus, the platform 110 may generate texture heatmaps 222 and/or other heatmaps 226 (e.g., based on various preprocessed images and/or raw hyperspectral images) at step 312.

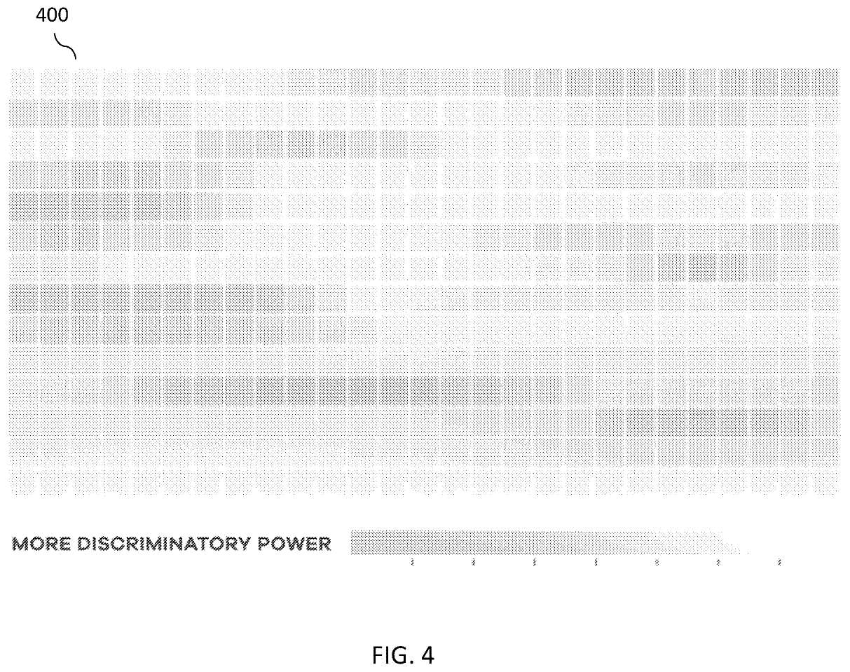

In some cases, the platform 110 may, as part of step 312 or otherwise, generate a visualization of any or all of the heatmaps 220 by plotting them. For instance, the platform 110 may plot a heatmap 220 using an appropriate color scheme, such as a jet color map, as shown in the example heatmap visualization 400 of FIG. 4A. Specifically, as shown in the example heat map visualization 400, the p-values and the color map range are bounded between 0 and 1, such that the varying pixel values are visually represented using different colors.

The platform 110 may store the heatmaps 220 and/or their corresponding visualizations in storage 116. The platform 110 may provide the stored visualizations for viewing and/or analysis by different user devices 130A-N and/or analysis systems 140A-N. For example, users and/or analysis systems may inspect the heatmaps (e.g., manually and/or using automated means) to determine their discriminatory power and/or which regions are most discriminatory. It should be noted that the platform 110 may employ other visualization approaches or color schemes when plotting the heatmaps.

At step 314, the platform 110 may rank the heatmaps 220 based on their importance, which may be measured by a score indicating a final discriminative power of each heat map. The platform 110 may calculate the final discriminative power using various methods. According to a first example method, the platform 110 may calculate a mean pixel discriminative power. Using this approach, for a single heatmap, the platform 110 may sum the pixel values of the heatmap and divide the sum by the total number of pixels within the heatmap to generate a value indicating the final discriminative power.

Alternatively, using a second example method, the platform 110 may calculate a mean of the squared pixel discriminative power for each heatmap 220. This approach is similar to the previous one, but the platform 110 may instead square the pixel values prior to summing them up and dividing the sum by the number of pixels. This platform 110 may use this method to give more weight to pixels with higher values, while reducing the importance of pixels with lower values. FIG. 4B illustrates an example heatmap visualization 410 showing the effect of squaring the p-values for each pixel.

Alternatively, using a third example method, the platform 110 may calculate the top-k mean pixel discriminative power. This approach may be appropriate when many of the pixels of the retinal image correspond to regions of the eye that do not significantly contribute to discriminating between positive and negative patients. The platform 110 may therefore improve the final discriminative power calculation by not including pixels for these regions in the calculation, which may introduce noise and/or otherwise reduce the quality of the calculation. The platform 110 may therefore perform the final discriminative power calculation using only the top k percent of pixels that are most discriminative. The platform 110 may ignore the remaining pixels, which may only add noise. FIG. 4C illustrates an example heatmap visualization 420 showing the effect of using the top-k p-values, with an example k parameter of 0.2. In this approach, k may be a tunable parameter that may be adjusted to best fit a particular dataset.

After the final discriminative power is generated for all heatmaps 220, the platform 110 ranks the heatmaps based on their importance as measured by their final discriminative power. Via the ranking, the platform 110 identifies the heatmaps that are most discriminative of the classification label, (e.g., the heat maps that most effectively distinguish between positive and negative examples in the case of binary classification).

At step 316, the platform 110 may select or reject different candidate features based on the ranking of the heatmaps 220 by discriminative power. The platform 110 may select the features that are most appropriate for training a machine learning (ML) algorithm. It should be noted that the ML algorithm may be trained to predict the presence or absence of the disease that corresponds to the reference classification label.

The platform 110 may reject a certain quantity of non-discriminatory features based on various hyperparameters. For example, the platform may discard features of certain texture types and/or wavelengths based on their corresponding heatmaps falling below a final discriminative power threshold. Additionally or alternatively, the platform may discard certain pixels of certain texture types and/or wavelengths based on the individual pixels of a corresponding heatmap having low discriminative power values. Consequently, the platform may select subsets of pixels of various heatmaps that are most discriminative in order to find corresponding candidate features. These selected pixels may correspond to selected input features for the ML algorithm based on the type of each heat map (e.g., texture type and/or wavelength).

The platform 110 therefore preselects retinal hyperspectral image features on a per-pixel basis for various wavelengths and/or texture image types. Additionally or alternatively, the platform 110 may select retinal regions of significant interest as input features. Accordingly, the platform 110 automates the selection of features, avoiding the need for human intervention by generating heatmaps of the per-pixel discriminative values of image-based features. In other words, the platform 110 uses the heatmaps to infer a ranking of feature importance, thereby improving the quality of input features for ML algorithms while reducing the input dimension. Here, reducing the input dimension may refer to focusing on certain pixels or regions that are found to be discriminative of a particular disease or condition. This feature selection process enhances the ability of an ML model to detect the condition in question through improved training and inference.

At step 318, the platform 110 trains the machine learning (ML) model using the selected candidate features from a training data set. The platform may train any type of classification model or other appropriate ML model to predict the presence or absence of the disease. During the training (and at runtime), the inputs to the ML model may include the selected candidate features (e.g., the pixels that were found to be most discriminative) along with relevant metadata from the training dataset. As described above, the training datasets may include raw hyperspectral images and/or texture images that are generated based on the hyperspectral images, various metadata including demographic information (e.g., age, gender, ethnicity), anatomical information such as vessel morphometrics (e.g., tortuosity of arteries and veins, arteriole-to-venule ratio (AVR), fractal dimension), measurements of eye movement (e.g., micro saccades), among other retinal image and/or related data.

During the training process, the platform 110 may use the classification label from the training dataset to provide a loss function. The platform 110 may use various machine learning techniques, such as backpropagation and gradient descent, to improve the performance of the ML model based on the loss function. The platform 110 may use these ML techniques, among others, to train classification models to predict the presence or absence of the disease in question (and/or degrees of the disease, probabilities associated with a predicted diagnosis, etc.).

At step 320, the platform 110 may deploy the trained machine learning (ML) model to any system for use in the detection of diseases or other conditions based on captured retinal images and/or hyperspectral images of a patient's retinas combined with other relevant metadata provided as input to the ML model. In some embodiments, the platform 110 may deploy the ML model to the analysis systems 140A-N and/or user devices 130A-N, which may use selected pixels of images captured by various retinal cameras (and/or selected pixels of texture images generated therefrom) as inputs to the ML model, enabling it to predict the presence or absence of the condition in the patient.

Additionally or alternatively, the platform 110 (rather than some other system) may use the ML model to implement an automated diagnostic system. For instance, the platform 110 may receive patient image data from the retinal cameras 120 and use selected pixels of the patient images (and/or texture images generated therefrom) to generate diagnostic predictions using the trained ML model.

The platform 110 may continue to train and/or fine-tune the ML model to enhance its performance as the training dataset grows and/or as the better candidate features are selected (e.g., by retrying the process of FIG. 3 using different hyperparameters). Accordingly, the platform 110 may repeatedly execute the process of FIG. 3 to improve a performance of the ML model.

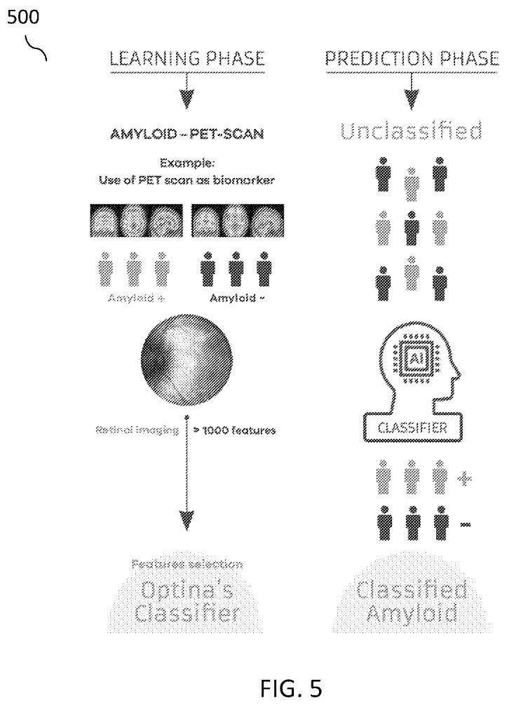

FIG. 5 illustrates data flow diagram 500 that may be implemented by the platform 110. The diagram 500 provides another example of the generation and use of texture heatmaps 222 as described above. At step 510, the platform 110 may generate a plurality of texture heatmaps 222. As shown in the figure, the generation of the texture heatmaps 222 may begin with flipping, padding and/or otherwise aligning a plurality of texture images as described above for steps 306-308. Then, at step 514, the platform 110 may combine the flipped/padded/aligned images into a single image stack. Next, the platform 110 may perform a number N of pixel-wise t-tests on various subsets of the image stack to generate a number N of texture heatmaps 222.

Next, at step 520, the platform 110 may perform feature ranking of the heatmaps based on a discriminative power of each heatmap, as described above for step 314. For example, at step 522 the platform 110 may use the feature ranking to select the top k features, where each feature is a pixel of a corresponding heatmap. Then, at step 530, the platform 110 may use the selected features to train the ML model.

FIG. 6 illustrates an example method for generating feature heatmaps 224, which may be used in addition to or instead of the texture heatmaps 222 for any of the uses described herein. At step 602, the platform 110 retrieves a hyperspectral image data set 200 and, at step 604, the platform 110 generates a texture image data set. The platform 110 may perform these steps as described above for steps 302 and 304, including any preprocessing, such as normalization and/or registration.

At step 606, the platform 110 may apply one or more anatomical masks to the various images (e.g., the hyperspectral images and/or texture images). Each anatomical mask may be a template that is used to isolate and identify specific anatomical structures or regions within a retinal image. These anatomical structures may include the blood vessels, optic nerve head, background retina, perivascular region, peripapillary region, and/or macular region, among others. The platform 110 may apply an anatomical mask by overlaying the mask onto a retinal image so that the features corresponding to the mask are highlighted or isolated. The platform 110 may apply the masks using image processing techniques such as segmentation. For example, the platform 110 may apply a mask for blood vessels in order to generate an image that primarily shows blood vessels, thus making it easier to analyze the blood vessels by simplifying the image and focusing the image information on particular features.

At step 608, the platform 110 may select particular spectral regions. For example, the platform 110 may select a number of spectral bands depending on which wavelengths within the hyperspectral data are associated with particular data features such as peaks, valleys, and/or other spectral features. Thus, the platform 110 may divide and/or segment the data into spectral regions, where the spectral regions may be isolated for further analysis.

At step 610, the platform 110 may extract the various features from the hyperspectral images and/or texture data (e.g., based on the anatomical masks, spectral data, and/or texture types). Each feature may include a value or set of values that represents a particular combination of an anatomical mask, spectral region, and/or texture measure. The platform 110 may generate hundreds or thousands of features based on different combinations. In some cases, the features may be measurements of, for example, the size, shape, and/or color of certain structures in the various images generated for the various features, vessel morphometrics (e.g., tortuosity of arteries and veins, arteriole-to-venule ratio (AVR), vessel width, fractal dimension), and/or any other features.

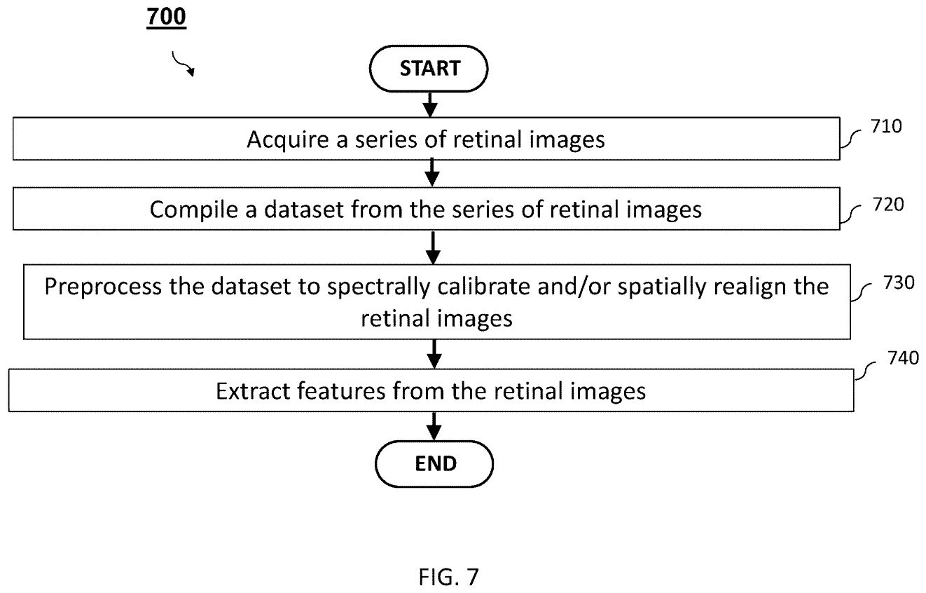

At step 612, the platform 110 may generate a feature grid based on the extracted features. An example feature heatmap 700 generated based on the feature grid is shown at FIG. 7. Although the feature heatmap 700 is not generated until step 614, the feature grid may have the same structure (but may store feature data instead of data that indicates discriminative power of the feature data, as shown in FIG. 7). In the example grid/heatmap, the vertical axis of the grid/heatmap may represent different non-spectral features, with each row of the heatmap corresponding to a different non-spectral feature. The horizontal axis may represent a different wavelength range for each column-thus in the example grid/heatmap, each box represents a different feature, with each row providing a number of different features that differ spectrally by column.

At step 614, the platform 110 may generate the heatmap 700 including a value representative of discriminative power using a statistical test to determine the discriminative power of each feature based on a reference label for a plurality of patients, as described above. In other words, the platform 110 may generate the feature heatmap 224 in a similar manner using a statistical feature-wise test, but where each feature is treated like a pixel for the pixel-wise test as described above (e.g., data for each feature is divided into two distributions based a reference label and the test measures the discriminative power of the feature).

From there, the platform 110 may continue, at step 616, to select and/or reject candidate features based on discriminative power as described above for step 316, train an ML model at step 618 as described above for step 318, and/or deploy a trained ML model 620 as described for step 320.

Although the methods of FIGS. 3 and 6 are described as being separate methods, the methods may be combined so that the platform 110 may generate texture heatmaps 222 at the same time or a different time that it generates the feature heatmaps 224 (e.g., with steps 306-314 occurring in series and/or in parallel with steps 606-614), and may use the feature heatmaps 224 together with the texture heatmaps (e.g., in a single ranking of both the texture heatmaps 222 and feature heatmaps 224 at step 314). Accordingly, any of the various types of heatmaps described herein can be used interchangeably for feature preselection, training of ML models, inference using trained ML models for diagnostics, and/or for any other purpose described herein.

The data transferred to and from various computing devices in the environment 100 may include secure and sensitive data, including personally identifiable information and patient data. Therefore, it may be desirable to protect transmissions of such data using secure network protocols and encryption, and/or to protect the integrity of the data when stored on the various computing devices. For example, a file-based integration scheme or a service-based integration scheme may be utilized for transmitting data between the various computing devices. Data may be transmitted using various network communication protocols. Secure data transmission protocols and/or encryption may be used in file transfers to protect the integrity of the data, for example, File Transfer Protocol (FTP), Secure File Transfer Protocol (SFTP), and/or Pretty Good Privacy (PGP) encryption. In many embodiments, one or more web services may be implemented within the various computing devices. Web services may be accessed by authorized external devices and users to support input, extraction, and manipulation of data between the various computing devices in the environment 100. Web services built to support a personalized display system may be cross-domain and/or cross-platform and may be built for enterprise use. Data may be transmitted using the Secure Sockets Layer (SSL) or Transport Layer Security (TLS) protocol to provide secure connections between the computing devices. Web services may be implemented using the WS-Security standard, providing for secure SOAP messages using XML encryption. Specialized hardware may be used to provide secure web services. For example, secure network appliances may include built-in features such as hardware-accelerated SSL and HTTPS, WS-Security, and/or firewalls. Such specialized hardware may be installed and configured in the environment 100 in front of one or more computing devices such that any external devices may communicate directly with the specialized hardware.

FIG. 8 illustrates an example platform 110 including exemplary hardware components. In embodiments, the platform 110 may include one or more processor(s) 802 for controlling overall operation of the platform 110 and its associated components, including memory(s) 804, network interface(s) 806, and/or input/output devices(s) 808. The memory(s) 808 may be non-transitory memories that may store computer-readable instructions that, when executed by the processor(s) 802, cause the platform to carry out the operations described herein. A data bus 810 may interconnect the processor(s), memory(s), I/O device(s), and/or network interface(s). In some embodiments, the platform 110 may represent, be incorporated in, and/or include various devices such as a desktop computer, a computer server, a mobile device, such as a laptop computer, a tablet computer, a smart phone, any other types of mobile computing devices, and the like, and/or any other type of data processing device.

Software may be stored within the memory 804 of the platform 110 to provide instructions to the processor(s) to allow the platform 110 to perform various actions. For example, the memory may store software used by the platform 110, such as an operating system, software for processing data and/or providing data to client devices, and an associated internal database (e.g., storage 116). The various hardware memory units in the memory may include volatile and nonvolatile, removable and non-removable media implemented in any method or technology for storage of information such as computer-readable instructions, data structures, program modules, or other data. The memory may include one or more physical persistent memory devices and/or one or more non-persistent memory devices. The memory may include, but is not limited to, random access memory (RAM), read only memory (ROM), electronically erasable programmable read only memory (EEPROM), flash memory or other memory technology, optical disk storage, magnetic cassettes, magnetic tape, magnetic disk storage or other magnetic storage devices, or any other medium that may be used to store the desired information and that may be accessed by the processor(s).

The network interface(s) 806 of the platform 110 may include one or more transceivers, digital signal processors, and/or additional circuitry and software for communicating via any network, wired or wireless, using any protocol as described herein.

The processor(s) 802 of the platform 110 may include a single central processing unit (CPU), which may be a single-core or multi-core processor or may include multiple CPUs. The processor(s) and associated components may allow the platform 110 to execute a series of computer-readable instructions to perform some or all of the processes described herein. Although not shown in FIG. 8, various elements within the platform 110 may include one or more caches, for example, CPU caches used by the processor(s), page caches used by the operating system, disk caches of a hard drive, and/or database caches used to cache content from a database. For embodiments including a CPU cache, the CPU cache may be used by one or more processors to reduce memory latency and access time. A processor may retrieve data from or write data to the CPU cache rather than reading/writing to memory, which may improve the speed of these operations. In some examples, a database cache may be created in which certain data from a database is cached in a separate smaller database in a memory separate from the database, such as in RAM or on a separate computing device. For instance, in a multi-tiered application, a database cache on an application server may reduce data retrieval and data manipulation time by not needing to communicate over a network with a back-end database server. These types of caches and others may be included in various embodiments and may provide potential advantages in certain implementations of devices, systems, and methods described herein, such as faster response times and less dependence on network conditions when transmitting and receiving data.

Although various components of the platform 110 are described separately, functionality of the various components may be combined and/or performed by a single component and/or multiple computing devices in communication.

The various systems, and devices described herein may have similar or different architecture as described with respect to the platform 110. Those of skill in the art will appreciate that the functionality of the platform 110 as described herein may be spread across multiple data processing devices, for example, to distribute processing load across multiple computers, to segregate transactions based on geographic location, user access level, quality of service (QOS), etc.

Referring to FIG. 9, there is shown a representation of a hyperspectral retinal camera 900 along with a schematic representation of a hyperspectral retinal dataset 902-906, in accordance with non-limiting embodiments of the present technology. The hyperspectral retinal camera 900 is an example of a retinal camera 120 as mentioned above. The hyperspectral retinal camera 900 is configured to operate as part of a phenotyping platform, such as, but not-limited to, the Optina Diagnostics' Retinal Deep Phenotyping™ platform from Optina Diagnostics (which may be implemented by the platform 110). In some embodiments, the phenotyping platform may be operated by a computer-implemented system, such as the system 1300 of FIG. 13 (which is an example implementation of the platform 110). The phenotyping platform may be configured to operate image analysis software tools for detection of phenotypic biomarkers of pathologies showing manifestations in the retina, including, but not-limited to, systemic diseases. As an example, the phenotyping platform may be used to reveal information that can help diagnose silent and under-detected systemic diseases affecting the 50+ population before irreversible damages occur.

The hyperspectral retinal camera 900 may be configured to sequentially capture a series of retinal images obtained at specific wavelengths (colors), for example under mydriatic conditions, while the patient sees a rapid rainbow of colors. It should be understood that this aspect is not limitative and other approaches may equally be envisioned, such as, capturing retinal images under non-mydriatic conditions. The spectral range covers the visible and near-infrared (NIR) spectra (450-905 nm), with images obtained in steps of 5 nm. The series of retinal images define the content of the hyperspectral retinal dataset 902-906. In some embodiments, the hyperspectral retinal dataset 902-906 may comprise, for example and without limitation, a series of 92 images on a 31-degree field of view of the retina, that may be obtained in less than one second. In some embodiments, the hyperspectral retinal dataset 902-906 may equally be referred to as a hyperspectral cube of data with two spatial dimensions and one spectral dimension. In some embodiments, the hyperspectral retinal dataset 902-906 may be the same as or part of the data set 200.

Once the acquisition is completed, the retinal hyperspectral cube is preprocessed with appropriate normalization and registration to spectrally calibrate and spatially realign images in order to correct for any eye movement that may have occurred during the image acquisition. The preprocessing therefore allows to have a continuous reflectance spectrum for each pixel of the image corresponding to a 8.6 μm×8.6 μm region of the retina (which encompasses a few retinal cells). While conventional fundus imaging techniques only yield structural information from three large spectral bands (red-green-blue), the present technologies allow the creation of images that provide deep spectral reflectance information from, for example and without limitation, over 90 bands of light, providing very rich datasets containing approximately 90 million pixels of data in a single image. It is as though, for each pixel of the image, a drawer containing information on the tissue composition in that location was made available. As can be seen from FIG. 10, the captured reflectance spectrum 1000 is influenced by the molecular content (e.g., hemoglobin, melanin), cellular arrangement (e.g., capillaries, nerve fiber layer), and/or density/thickness (e.g., neurodegeneration) of the tissue. Thus, in addition to anatomical structures, this spectral-rich information can be used to identify a wealth of phenotypic features based on anatomical and functional changes in the retinal tissue—including all its complex neurological and vascular structures—that correlate with systemic diseases. In the embodiment illustrated at FIG. 10, the reflectance spectrum is available from each pixel of the hyperspectral retinal image.

As can be seen from FIG. 11, in some embodiments, the phenotyping platform is configured to execute a computer-implemented method 1100, executing steps 1102-1108, allowing extraction of thousands of features from each retinal image, using different combinations of anatomical masks (e.g., vessels and their periphery, optic nerve head, background retina, etc.), spectral regions and texture measures. In some embodiments, the method 1100 may be executed as part of the method of FIG. 6 (e.g., steps 1102-1108 may be the same as or part of steps 602-610). Texture image analysis may be helpful to highlight subtle patterns in images that are invisible to the naked eye. The technique is particularly useful to mine both the spectral and spatial dimensions of the rich datasets captured with hyperspectral retinal camera 900, allowing for the extraction of different features. In some embodiments, thousands of features are extracted using a combination of anatomical masks, spectral regions, and/or texture measures. In some other embodiments, features may also be extracted from anatomical measures from the retinal images, such as, but not-limited to, vessel diameter, vessel tortuosity, vessel tree fractal dimension, optic nerve head diameter, optic nerve head cup to disc ration, etc. With respect to the usage of texture measures, further details may be found in U.S. Pat. No. 10,964,036, the content of which is hereby incorporated by reference.

In some embodiments, a feature grid is built from each of the individual features extracted from the hyperspectral retinal dataset 902-906, each square on the grid corresponding to a specific feature. The feature grid is also described above at step 612. A retinal heat map, such as the retinal heat map 700 shown at FIG. 7, can then be developed for each disease or condition of interest through the collection of a reference database, using a recognized reference biomarker (ground truth) to determine the presence or absence of the disease/condition in a representative patient population, some who have the given disease or condition and some who do not. The retinal heat map may be generated as described above for step 614. Retinal images are captured for every patient of this reference database and some or all features of the grid are computed for each one. The feature distribution is then compared between individuals showing the reference biomarker and those who do not show or express such phenotype.

In the embodiments shown at FIG. 7, the retinal deep phenotyping heat map 700 illustrates a discriminatory power of individual retinal features that may be developed for a given disease or condition from a subject database with and without the presence of a reference biomarker. The retinal deep phenotyping heat map 700 is generated by using a color scale indicating the discriminatory power of a feature to differentiate those with the reference biomarker from those without. Some features denoted by lighter areas on the heat map 700 have little or no discriminatory power, meaning that their values are very similar for those with and without the given condition. In other words, their distributions may strongly overlap. At the other end of the discriminatory scale, some other features denoted by darker areas on the heat map 700 display much less overlap between the groups with and without the reference biomarker and therefore show higher discriminatory power. The retinal deep phenotyping heat map 700 may therefore be useful to identify, at a glance, those spatial-spectral features that are most discriminatory for a given disease or condition that affects a condition of the retina and could readily be used to provide insights on the reference biomarker.

It should be understood that the embodiment of the retinal deep phenotyping heat map 700 of FIG. 7 is exemplary and therefore not limitative. Other embodiments may equally be envisioned, for example, by allowing interactions between a user and the retinal deep phenotyping heat map 700. The interaction may be operated by an interactive tool configured to allow the user to select and/or click on one or more features visually represented on the retinal deep phenotyping heat map 700, for example, the one or more features that may be more discriminatory than others. Upon selecting and/or clicking on one or more given features, the user may be presented with information relating to the one or more given features. Such information may comprise, for example, but without being limitative, type of texture, spectral interval, anatomic position associated with the one or more given features, etc.

In some embodiments, the retinal deep phenotyping heat map 700 may take the form a matrix wherein each one of the features is represented by a distinct square on the retinal deep phenotyping heat map 700. It should be understood that the specific geometrical shape of the feature representation is not limitative and other shapes may be envisioned, such as, for example, circles. In such an embodiment, the heat map take the form of a plate comprising circles (e.g., a virtual well plate configured in a similar way as the well plates often used in biology). Three-dimensional (3D) representations are also contemplated for example, through the displaying of cubes, spheres and so on.

Turning now to FIG. 12, an exemplary embodiment of a creation of a test 1200 is illustrated. In some embodiments, a test may be built for each targeted disease or condition. The test may be based on a machine-learning (ML) model of selected features from the retinal deep phenotyping heat map 700. The selected featured may for example have been extracted from retinal images and classified using the ML model in the manner described for example in United States Patent Application Publication No. US20220157470A1, published on May 19, 2022, the disclosure of which is incorporated by reference herein. The resulting classification model, based on a set of few features, can achieve better discriminatory power than those features considered individually and can be used as a diagnostic test for new patients to determine the reference biomarker's classification status (presence or absence) based solely on features extracted from the hyperspectral retinal dataset 902-906. In the illustrated embodiment, the test 1200 may be built from a selection of features and can then be used to detect the presence or absence of the reference biomarker from the hyperspectral retinal images alone.

As the skilled reader may appreciate, the present technology may allow exploiting the subtle retinal manifestations associated with systemic and ocular diseases. Datasets may be enhanced to generate heat maps and classification models for multiple underdiagnosed age-related diseases, including neurodegenerative diseases such as Alzheimer's, cardiovascular, and ophthalmic diseases and other systemic conditions.

Referring now to FIG. 13, a schematic diagram of a system 1300 is shown. The system 1300 may be the same as or different from the platform 110. For example, the system 1300 is one example embodiment of the platform 110. The system 1300 being suitable for implementing non-limiting embodiments of the present technology. It is to be expressly understood that the system 1300 as depicted is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 1300 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 1300 may provide in certain instances simple implementations of the present technology, and that where such is the case they have been presented in this manner as an aid to understanding. As persons skilled in the art would understand, various implementations of the present technology may be of a greater complexity.

Generally speaking, the system 1300 comprises a computing unit 1305. In some embodiments, the computing unit 1305 may be implemented by any of a conventional personal computer, a controller, and/or an electronic device (e.g., a server, a controller unit, a control device, a monitoring device etc.) and/or any combination thereof appropriate to the relevant task at hand. In some embodiments, the computing unit 1305 comprises various hardware components including one or more single or multi-core processors collectively represented by a processor 1310, a solid-state drive 1330, a RAM 1340, a dedicated memory 1350 and an input/output interface 1360. The computing unit 1305 may be a generic computer system.

In some other embodiments, the computing unit 1305 may be an “off the shelf” generic computer system. In some embodiments, the computing unit 1305 may also be distributed amongst multiple systems. The computing unit 1305 may also be specifically dedicated to the implementation of the present technology. As a person in the art of the present technology may appreciate, multiple variations as to how the computing unit 1305 is implemented may be envisioned without departing from the scope of the present technology.

Communication between the various components of the computing unit 1305 may be enabled by one or more internal and/or external buses 1370 (e.g. a PCI bus, universal serial bus, IEEE 1394 “Firewire” bus, SCSI bus, Serial-ATA bus, ARINC bus, etc.), to which the various hardware components are electronically coupled.

The input/output interface 1360 may provide networking capabilities such as wired or wireless access. As an example, the input/output interface 1360 may comprise a networking interface such as, but not limited to, one or more network ports, one or more network sockets, one or more network interface controllers and the like. Multiple examples of how the networking interface may be implemented will become apparent to the person skilled in the art of the present technology. For example, but without being limitative, the networking interface may implement specific physical layer and data link layer standard such as Ethernet, Fibre Channel, Wi-Fi or Token Ring. The specific physical layer and the data link layer may provide a base for a full network protocol stack, allowing communication among small groups of computers on the same local area network (LAN) and large-scale network communications through routable protocols, such as Internet Protocol (IP).

In some embodiments, the hyperspectral retinal camera 900 of FIG. 1 may be in communication with the computing unit 1305 via the one or more internal and/or external buses 1370 and/or the input/output interface 1360.

According to implementations of the present technology, the solid-state drive 1330 stores program instructions suitable for being loaded into the RAM 1340 and executed by the processor 1320. Although illustrated as a solid-state drive 1330, any type of memory may be used in place of the solid-state drive 1330, such as a hard disk, optical disk, and/or removable storage media.

The processor 1310 may be a general-purpose processor, such as a central processing unit (CPU) or a processor dedicated to a specific purpose, such as a digital signal processor (DSP). In some embodiments, the processor 1310 may also rely on an accelerator 1320 dedicated to certain given tasks, such as executing the methods set forth in the paragraphs below. In some embodiments, the processor 1310 or the accelerator 1320 may be implemented as one or more field programmable gate arrays (FPGAs). Moreover, explicit use of the term “processor”, should not be construed to refer exclusively to hardware capable of executing software, and may implicitly include, without limitation, application specific integrated circuit (ASIC), read-only memory (ROM) for storing software, RAM, and non-volatile storage. Other hardware, conventional and/or custom, may also be included.

Further, the system 1300 may include a screen or display 1306 capable of rendering an interface of the communication application and/or the one or more outputs 1380. In some embodiments, display 1306 may comprise and/or be housed with a touchscreen to permit users to input data via some combination of virtual keyboards, icons, menus, or other Graphical User Interfaces (GUIs). In some embodiments, display 1306 may be implemented using a Liquid Crystal Display (LCD) display or a Light Emitting Diode (LED) display, such as an Organic LED (OLED) display. The device may be, for example and without being limitative, a handheld computer, a personal digital assistant, a cellular phone, a network device, a smartphone, a navigation device, an e-mail device, a game console, or a combination of two or more of these data processing devices or other data processing devices.

The system 1300 may comprise a memory 1330 communicably connected to the computing unit 1305 for storing the one or more outputs 608 for example. The memory 1330 may be embedded in the system 1300 as in the illustrated embodiment of FIG. 13 or located in an external physical location. The computing unit 1305 may be configured to access a content of the memory 1330 via a network (not shown) such as a Local Area Network (LAN) and/or a wireless connexion such as a Wireless Local Area Network (WLAN).

The system 1300 may also includes a power system (not depicted) for powering the various components. The power system may include a power management system, one or more power sources (e.g., battery, alternating current (AC)), a recharging system, a power failure detection circuit, a power converter or inverter and any other components associated with the generation, management and distribution of power in mobile or non-mobile devices.

FIG. 14 is a flow diagram of a method 1400 for processing retinal images, according to some embodiments of the present technology.

In one or more aspects, the method 1400 or one or more steps thereof may be performed by a processor or a computer system, such as the computing unit 1305 and/or processor(s) 802. The method 1400 or one or more steps thereof may be embodied in computer-executable instructions that are stored in a computer-readable medium, such as a non-transitory mass storage device, loaded into memory and executed by a CPU. Some steps or portions of steps in the flow diagram may be omitted or changed in order.

The method 1400 includes, at step 1410, acquiring a series of retinal images. In some embodiments, the acquiring is operated at specific wavelengths, for example under mydriatic or non-mydriatic conditions. The spectral range covers the visible and near-visible spectra. The spectral range of the wavelengths varies from (450-905 nm). The retinal images are obtained in steps of 5 nm.

The method 1400 includes, at step 1420, compiling a dataset from the series of retinal images. In some embodiments, the dataset comprise a series of 92 images on a 31-degree field of view of the retina.

The method 1400 includes, at step 1430, preprocessing the dataset to spectrally calibrate and/or spatially realign the retinal images. In some embodiments, the preprocessing allows creation of a reflectance spectrum from each pixel of the retinal images.

The method 1400 includes, at step 1440, extracting features from the retinal images. In some embodiments, the extracting is based on a combination of anatomical masks, spectral regions and/or texture measures. In the same or other embodiments, the ML model may be used to classify the extracted features.

In some embodiments, the method 1400 further comprises the computing of a feature grid from the extracted features. In some embodiments, the feature grid comprises squares, each one of the squares corresponding to a specific feature. In some embodiments, the method 1400 further comprises the computing of an heat map from the feature grid. The heat map is associated with a given disease or condition of interest through the collection of a reference database, using a recognized reference biomarker to determine the presence or absence of the disease or condition in a representative patient population.

In some embodiments, the method 1400 may comprise, in combination with or instead of the steps 1410-1440, the steps of displaying, to a user, a feature grid; receiving, from the user, an input to select a feature from the feature grid; and displaying, to the user, information relating to the selected feature. In some embodiments, the information comprises at least one of a type of texture, a spectral interval and/or an anatomic position associated with the one or more given features. In some embodiments, the feature grid is represented as a matrix comprising squares, each one of the squares being associated with a distinct feature. In some alternative embodiments, the feature grid is represented as a plate comprising circles, each one of the circles being associated with a distinct feature.

One or more aspects discussed herein may be embodied in computer-usable or readable data and/or computer-executable instructions, such as in one or more program modules, executed by one or more computers or other devices as described herein. Generally, program modules include routines, programs, objects, components, data structures, and the like. that perform particular tasks or implement particular abstract data types when executed by a processor in a computer or other device. The modules may be written in a source code programming language that is subsequently compiled for execution or may be written in a scripting language such as (but not limited to) HTML or XML. The computer executable instructions may be stored on a computer readable medium such as a hard disk, optical disk, removable storage media, solid-state memory, RAM, and the like. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a system, and/or a computer program product.

A first computer-implemented method for processing retinal images is disclosed. The method may comprise acquiring a series of retinal images, compiling a dataset from the series of retinal images, preprocessing the dataset to spectrally calibrate and/or spatially realign the retinal images, and extracting features from the retinal images.

In some embodiments, the first method further comprises the computing of a feature grid from the extracted features.

In embodiments of the first method, the feature grid comprises squares, each one of the squares corresponding to a specific feature.

In some embodiments, the first method further comprises the computing of a heat map from the feature grid.

In embodiments of the first method, the heat map is associated with a given disease or condition of interest through the collection of a reference database, using a recognized reference biomarker to determine the presence or absence of the disease or condition in a representative patient population.

In some embodiments, the first method further comprises displaying, to a user, the feature grid, receiving, from the user, an input to select a feature from the feature grid, and displaying, to the user, information relating to the selected feature.

In embodiments of the first method, the information comprises at least one of a type of texture, a spectral interval and/or an anatomic position associated with the one or more given features.

In embodiments of the first method, the feature grid is represented as a matrix comprising squares, each one of the squares being associated with a distinct feature.

In embodiments of the first method, the feature grid is represented as a plate comprising circles, each one of the circles being associated with a distinct feature.

In embodiments of the first method, the acquiring is operated at specific wavelengths under mydriatic conditions.

In embodiments of the first method, the spectral range covers the visible and near-visible spectra.

In embodiments of the first method, the spectral range of the wavelengths varies from (450-905 nm).

In embodiments of the first method, the retinal images are obtained in steps of 5 nm.

In embodiments of the first method, the dataset comprise a series of 92 images on a 31-degree field of view of the retina.

In embodiments of the first method, the preprocessing allows creation of a reflectance spectrum from each pixel of the retinal images.

In embodiments of the first method, the extracting is based on a combination of anatomical masks, spectral regions and/or texture measures.

In some embodiments, the first method further comprises using a machine learning model to classify the extracted features.

A second computer-implemented method for representing features is disclosed. The method may comprise displaying, to a user, a feature grid, receiving, from the user, an input to select a feature from the feature grid, and displaying, to the user, information relating to the selected feature.

In embodiments of the second method, the information comprises at least one of a type of texture, a spectral interval and/or an anatomic position associated with the one or more given features.

In embodiments of the second method, the feature grid is represented as a matrix comprising squares, each one of the squares being associated with a distinct feature.

In embodiments of the second method, the feature grid is represented as a plate comprising circles, each one of the circles being associated with a distinct feature.

A system for processing retinal images is disclosed. The system may comprise a controller and a memory storing a plurality of executable instructions which, when executed by the controller, cause the system to perform any of the embodiments of the first method and the second method.

A non-transitory computer-readable medium comprising computer-readable instructions is disclosed. The computer-readable instructions, upon being executed by a system, may cause the system to perform any of the embodiments of the first method and the second method.

Although the present disclosure has been described using certain specific aspects, many additional modifications and variations would be apparent to those skilled in the art. In particular, any of the various processes described above may be performed in alternative sequences and/or in parallel (on different computing devices) in order to achieve similar results in a manner that is more appropriate to the requirements of a specific application. It is therefore to be understood that the techniques described herein may be practiced otherwise than specifically described. Thus, embodiments should be considered in all respects as illustrative and not restrictive. Accordingly, the scope of the invention should be determined not by the embodiments illustrated, but by the appended claims and their equivalents.

1. A method comprising:
receiving a plurality of retinal images corresponding to a plurality of patients;
modifying at least some of the plurality of retinal images so that each of the plurality of retinal images shares an orientation;
generating, based on the modified plurality of retinal images, a stack of retinal images for further analysis, where each retinal image of the stack of retinal images comprises a defined number of pixels, wherein each retinal image of the stack of retinal images is associated with at least one reference label indicating a presence or absence of a medical condition;
for each pixel of the defined number of pixels:
calculating a first distribution value for the corresponding pixel, wherein the first distribution value is based on pixel values from a first subset of retinal images associated with a positive reference label for the medical condition;
calculating a second distribution value, wherein the second distribution value is based on pixel values from a second subset of retinal images associated with a negative reference label for the medical condition; and
calculating a comparison metric for the corresponding pixel based on the first distribution value and the second distribution value;
generating a heat map based on the comparison metric for each pixel of the defined number of pixels; and
calculating a discriminative power of the heat map for detecting the medical condition.

2. The method of claim 1, wherein the heat map is a first heat map associated with a first spectral range and a first texture type, further comprising:
generating a plurality of heat maps, wherein each heat map is associated with a spectral range and a texture type, wherein the plurality of heat maps includes the first heat map;
calculating a discriminative power for each of the plurality of heat maps; and
ranking the plurality of heat maps based on the corresponding discriminative power of each of the plurality of heat maps.

3. The method of claim 2, further comprising selecting one or more features to train a machine learning model for detecting the medical condition based on the ranking of the plurality of heatmaps.

4. The method of claim 1, wherein calculating the discriminative power of the heat map comprises calculating a mean of each comparison metric of the heat map.

5. The method of claim 1, wherein calculating the discriminative power of the heat map comprises calculating a mean of each squared comparison metric of the heat map.

6. The method of claim 1, wherein calculating the discriminative power of the heat map comprises calculating a mean of a sub-set of top-k comparison metrics of the heat map.

7. The method of claim 1, wherein modifying the subset of the plurality of retinal images comprises flipping the subset of retinal images that correspond to a left or right eye so that they share the orientation of a right or left eye.

8. The method of claim 1, wherein modifying the subset of the plurality of retinal images comprises padding each retinal image to center an optical nerve head within each of the plurality of retinal images.

9. The method of claim 1, wherein the calculating of the first distribution value, second distribution value, and comparison metric is performed as part of a Student's t-test.

10. The method of claim 1, wherein generating the stack of retinal images further comprises performing non-linear image registration to align retinal anatomical landmarks associated with each of the plurality of patients.

11. A method comprising:
generating a stack of retinal images corresponding to a plurality of patients, wherein each retinal image of the stack of retinal images is aligned and comprises a defined number of pixels, wherein each retinal image of the stack of retinal images is associated with at least one reference label indicating the presence or absence of a medical condition;
generating a first heat map for a first subset of images from the stack of retinal images by:
for each pixel of the defined number of pixels of the first subset of images:
calculating a first distribution value for the corresponding pixel, wherein the first distribution value is based on pixel values from a first subset of retinal images associated with a positive reference label for the medical condition;
calculating a second distribution value for the corresponding pixel, wherein the second distribution value is based on pixel values from a second subset of retinal images associated with a negative reference label for the medical condition; and
calculating a comparison metric for the corresponding pixel based on the first distribution value and the second distribution value;
generating the heat map for the first subset of images based on the comparison metric for each pixel of the defined number of pixels; and
generating additional heat maps for additional subsets of images from the stack of retinal images to yield a plurality of heat maps, wherein the plurality of heat maps comprises the first heat map;
determining a discriminative power of each heat map of the plurality of heat maps; and
ranking the plurality of heat maps based on the corresponding discriminative power of each of the plurality of heat maps.

12. The method of claim 11, further comprising:
selecting a plurality of features corresponding to pixels of the heat maps that have high discriminative power for the medical condition; and
training an ML model to predict the absence or presence of the medical condition based on the selected plurality of features.

13. The method of claim 11, wherein calculating the discriminative power of each heat map comprises, for each heat map, calculating a mean of each comparison metric.

14. The method of claim 11, wherein calculating the discriminative power of each heat map comprises, for each heat map, calculating a mean of each squared comparison metric of the heat map.

15. The method of claim 11, wherein calculating the discriminative power of the heat map comprises calculating a mean of a sub-set of top-k comparison metrics of the heat map.

16. The method of claim 11, wherein generating the stack of retinal images comprises flipping a subset of retinal images that correspond to a left or right eye so that they share an orientation of a right or left eye.

17. The method of claim 11, wherein generating the stack of retinal images comprises padding each image of the stack of retinal images to align an optical nerve head within each image.

18-19. (canceled)

20. A method comprising:
generating a data set of retinal images corresponding to a plurality of patients, wherein each retinal image of the stack of retinal images is aligned, wherein each retinal image of the stack of retinal images is associated with at least one reference label indicating the presence or absence of a medical condition;
generating texture measures for the data set of retinal images;
applying a plurality of anatomical masks to the data set of retinal images;
selecting spectral regions;
generating a plurality of features, wherein each feature corresponds to a particular texture measure, anatomical mask, and spectral region;
generating values for each of the plurality of features from the data set of retinal images to yield a feature grid comprising feature values for each of the plurality of features;
generating a heatmap based on the feature grid and a classification label indicating the presence or absence of a medical condition; and
measuring a discriminative power of the heatmap.

21.-24. (canceled)

25. A system for processing retinal images, the system comprising a processor and a memory storing a plurality of executable instructions which, when executed by the processor, cause the system to perform the method of claim 1.

26. A non-transitory computer-readable medium comprising computer-readable instructions that, upon being executed by a system, cause the system to perform the method claim 1.
